(19) United States
(12) Patent Application Publication
(10) Pub. No.: US 2022/0096336 A1
(43) Pub. Date: Mar. 31, 2022

(54) PEPTIDE MICROPARTICLE STRUCTURE

(71) Applicant: McCTILL Co., Ltd., Taipei City (TW)

(72) Inventors: JEFFRY HORNG CHIEH YEN, Taipei City (TW); TI HAO CHANG, Taipei City (TW)

(21) Appl. No.: 17/476,911

(22) Filed: Sep. 16, 2021

Related U.S. Application Data
(60) Provisional application No. 63/083,164, filed on Sep. 25, 2020.

(51) Int. Cl. A61K 8/02; A61K 8/11; A61K 8/9711; A61K 8/9789; A61K 8/9794; A61K 8/73; A61Q 19/08

(52) U.S. Cl. CPC A61K 8/0245; A61K 8/11; A61K 8/9711; A61K 8/9789; A61K 2800/622; A61K 8/735; A61Q 19/08; A61K 2800/652; A61K 2800/63; A61K 8/9794

(57) ABSTRACT

The disclosure provides a peptide microparticle structure that can easily penetrate into the bottom layer of the skin. The peptide microparticle structure is composed, from inside to outside, of a main core, a first lipid layer, an active ingredient layer, and a second lipid layer. The main core is disposed at the center of the peptide microparticle structure, and the outer part of the main core is completely covered by the first lipid layer. The outermost layer of the peptide microparticle structure is the second lipid layer, and between the first lipid layer and the second lipid layer is the active ingredient layer.

### BACKGROUND

### 1. Technical Field

The present disclosure relates to a microparticle structure, especially a microparticle structure that can easily penetrate into the bottom layer of the skin. The microparticle structure contains peptides that can promote the production of collagen and elastin.

### 2. Description of the Related Art

Peptides and proteins are both biochemical molecules composed of amino acids. The amino group (—NH₂) of an amino acid is dehydrated and condensed with the carboxyl group (—COOH) of another amino acid to form a peptide bond, thereby linking more peptide bonds to form a chain molecular structure. Generally, peptides refer to molecules composed of 2 to 50 amino acids, while proteins refer to molecules composed of more than 50 amino acids. Amino acids are an important element of biological composition. There are 20 common amino acids in the human body. For example, Glycine is an essential element for the normal function of the nervous system, skin, and muscle tissue; Alanine improves immune system function, assists wound healing, stabilizes cell structure, and maintains DNA integrity; and Proline is one of the important amino acids for the synthesis of human protein, which can speed up wound healing.

Peptides are named based on the number of the composed amino acids. Dipeptides are fragments composed of 2 amino acids, tripeptides are fragments composed of 3 amino acids, and so on. Different peptides have different physiological functions. For example, dipeptides improve lymphatic circulation. Tripeptides effectively prevent neurotransmission of acetylcholine to induce muscle relaxation, thereby reducing dynamic wrinkles, such as forehead lines, frown lines, nasolabial folds, smile lines, etc. Tetrapeptides control inflammatory response of the skin, reduce sagging skin, maintain the elasticity of skin, and create skin lifting effect. The structure of pentapeptides is similar to the precursors of type I collagen, which stimulates cells to synthesize the main components of extracellular matrix, such as collagen, elastin, hydration gel, etc., so pentapeptides reduce wrinkles, tighten the skin, and increase fullness and elasticity of the skin. Hexapeptides function similarly to botulinum toxin, which blocks transmission between muscles and nerves, smooths the skin lines, and has an anti-wrinkle lifting effect.

Since peptides have the above-mentioned functions, it will be great potential if they are used in human skin care. Therefore, it is urgent to solve the problem of how to make these peptides be effectively absorbed and utilized by human skin.

### SUMMARY

In order to make human skin effectively absorb peptides and fully utilize different physiological properties of various peptides, in one aspect, the present disclosure provides a peptide microparticle structure for packaging and accommodating one or more peptides. The structure comprises a main core located at the center of the peptide microparticle structure, wherein the main core contains one or more peptides; a first lipid layer disposed on the outside of the main core to completely cover the main core; a second lipid layer disposed on the outer surface of the peptide microparticle structure; and an active ingredient layer located between the first lipid layer and the second lipid layer.

Preferably, the peptide of the peptide microparticle structure of the present disclosure is selected from one of amino acid monomer molecules, tripeptides, tetrapeptides, pentapeptides, hexapeptides, and any combination thereof.

Preferably, the active ingredient layer of the peptide microparticle structure of the present disclosure contains one or more active ingredients, wherein the active ingredient is selected from the group consisting of edelweiss extract, strawflower extract, evening primrose extract, sea fennel extract, Rainbow algae extract, chamomile extract, oat extract, hyaluronic acid, and any combination thereof.

Preferably, the first lipid layer of the peptide microparticle structure of the present disclosure is made of at least one lipid.

Preferably, the second lipid layer of the peptide microparticle structure of the present disclosure is made of at least one lipid.

Preferably, the lipid in the peptide microparticle structure of the present disclosure consists of one of lecithin, phospholipid choline, cholesterol, or any combination thereof.

Preferably, the first lipid layer of the peptide microparticle structure of the present disclosure has a monolayer membrane structure or a bilayer membrane structure.

Preferably, the second lipid layer of the peptide microparticle structure of the present disclosure has a monolayer membrane structure or a bilayer membrane structure.

Preferably, the size of the peptide microparticle structure of the present disclosure is 50-500 nm.

The technical features and advantages of the present disclosure and its achievable effects will be illustrated by the following examples in combination with the drawings, so that those having ordinary skill in the art can understand the spirit of the disclosure and implement the disclosure accordingly. Any simple changes and modifications in the embodiments of the disclosure can be carried out without departing from the concept and scope of the disclosure.

### BRIEF DESCRIPTION OF THE DRAWINGS

FIG. 1 is a schematic cross-sectional view of the peptide microparticle structure according to an embodiment of the disclosure;

FIG. 2 is a schematic diagram of the monolayer membrane structure composed of lipid according to an embodiment of the disclosure; and

FIG. 3 shows the bilayer membrane structure composed of lipid according to an embodiment of the disclosure.

### DETAILED DESCRIPTION OF THE EMBODIMENTS

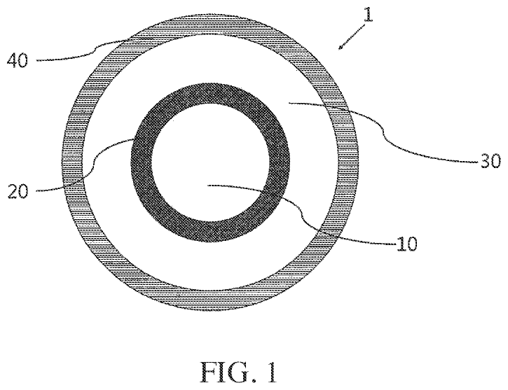

Please refer to FIG. 1, which is a schematic diagram of the peptide microparticle structure of an embodiment of the present disclosure. The peptide microparticle structure 1 is composed, from inside to outside, of a main core 10, a first lipid layer 20, an active ingredient layer 30, and a second lipid layer 40. The main core 10 is disposed at the center of the peptide microparticle structure 1, and the outer part of the main core 10 is completely covered by the first lipid layer 20. The outermost layer of the peptide microparticle structure 1 is the second lipid layer 40, and between the first lipid layer 20 and the second lipid layer 40 is the active ingredient layer 30.

The main core 10 is a accommodating space completely covered by the first lipid layer 20, and the accommodating space can be used to accommodate different types of peptides, which are selected from, but are not limited to, one of amino acid monomer molecules, tripeptides, tetrapeptides, pentapeptides, hexapeptides, and any combination thereof. All of the above peptides can be used for skin repair or skin care. For example, dipeptides improve lymphatic circulation. Tripeptides effectively prevent neurotransmission of acetylcholine to induce muscle relaxation, thereby reducing dynamic wrinkles. Tetrapeptides control inflammatory response of the skin, reduce sagging skin, maintain the elasticity of skin, and create skin lifting effect. Pentapeptides stimulate cells to synthesize the main components of extracellular matrix, such as collagen, elastin, and hydration gel, to reduce wrinkles, tighten the skin, and increase fullness and elasticity of the skin. Hexapeptides function similarly to botulinum toxin, which blocks transmission between muscles and nerves, smooths the skin lines, and has an anti-wrinkle lifting effect.

In one embodiment, the biological properties of the above-mentioned peptides are stable and maintained in the main core 10 encapsulated by the first lipid layer 20, so that the peptides accommodated in the peptide microparticle structure 1 of the present disclosure can be stored, transmitted, and released in a stable state.

Another accommodating space can be created between the first lipid layer 20 and the second lipid layer 40, and the accommodating space is the active ingredient layer 30. The active ingredient layer 30 can contain different kinds of active ingredients, which are selected from, but are not limited to, one of edelweiss extract, strawflower extract, evening primrose extract, sea fennel extract, Rainbow algae extract, chamomile extract, oat extract, hyaluronic acid, and any combination thereof. The above-mentioned active ingredients have the functions of moisturizing, anti-inflammatory, anti-oxidation, reducing wrinkles and spots, and enhancing the effect of skin repair or skin care.

In one embodiment, the chemical properties of the above-mentioned active ingredients are stable and maintained in the active ingredient layer 30, so that the active ingredients accommodated in the peptide microparticle structure 1 of the present disclosure can be stored, transmitted, and released in a stable state.

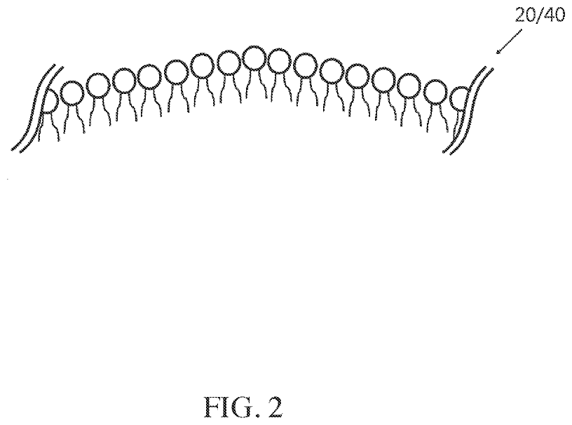

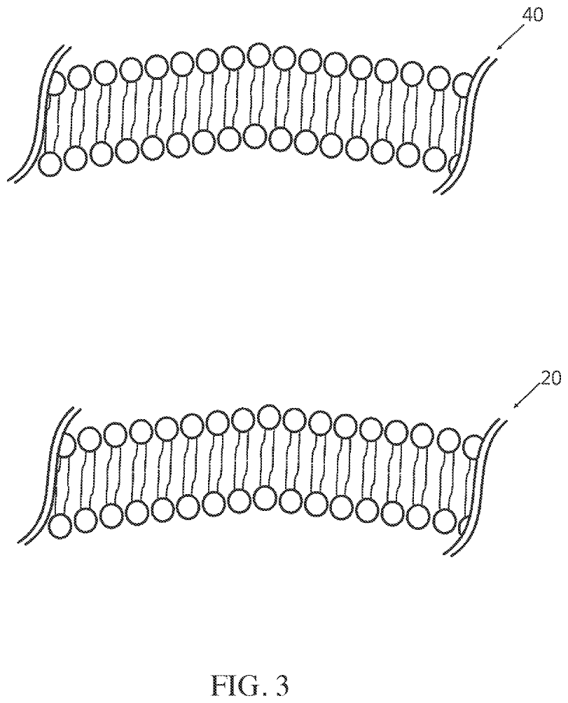

The first lipid layer 20 and the second lipid layer 40 are made of a lipid, which is, but not limited to, one of lecithin, phospholipid choline, cholesterol, or any combination thereof. With different reaction conditions, the lipid can form a monolayer membrane structure as shown in FIG. 2 or a bilayer membrane structure as shown in FIG. 3.

In one embodiment, the first lipid layer 20 and the second lipid layer 40 are both monolayer membrane structures composed of the lipid. In another embodiment, the first lipid layer 20 and the second lipid layer 40 are both bilayer membrane structures composed of the lipid.

In another embodiment, the first lipid layer 20 is a monolayer membrane structure composed of the lipid, and the second lipid layer 40 is a bilayer membrane structure composed of the lipid. In other embodiments, the first lipid layer 20 is a bilayer membrane structure composed of the lipid, and the second lipid layer 40 is a monolayer membrane structure composed of the lipid.

The size of the peptide microparticle structure 1 of the present disclosure is between 50-500 nm, and the peptide microparticle structure 1 can be used in skin care products, such as serum, lotion, and cream.

In summary, using the peptide microparticle structure of this disclosure as a carrier for peptides and active ingredients can help the peptides and active ingredients contained in the microparticle structure to penetrate into the bottom layer of human skin, making them easy to be absorbed and utilized by the skin, enhancing the production of collagen and elastin in the dermis of the skin, and creating the effects of moisturizing and whitening at the same time.

For the above composition and technical content of the peptide microparticle structure of the present disclosure, various applicable examples are listed and described in detail with reference to the accompanying drawings. However, the present disclosure is of course not limited to the listed embodiments, drawings, or detailed descriptions.

Those who are familiar with the technology of the invention should also understand that the listed embodiments and the accompanying drawings are only for reference and explanation, and are not intended to limit the disclosure. Inventions that can be completed with modifications or changes easily implemented based on the description are also deemed to be within the scope of the spirit and purpose of the present disclosure. Of course, these inventions are also included in the claim of the present disclosure.

What is claimed is:

1. A peptide microparticle structure, comprising:
a first lipid layer;
a main core located at the center of the peptide microparticle structure, the main core being an accommodating space completely covered by the first lipid layer, and the main core accommodating one or more peptides;
a second lipid layer disposed on the outer surface of the peptide microparticle structure; and
an active ingredient layer located between the first lipid layer and the second lipid layer.

2. The peptide microparticle structure of claim 1, wherein the peptide is one selected from the group consisting of amino acid monomer molecules, tripeptides, tetrapeptides, pentapeptides, hexapeptides, and any combination thereof.

3. The peptide microparticle structure of claim 1, wherein the active ingredient layer contains one or more active ingredients, wherein the active ingredient is selected from the group consisting of edelweiss extract, strawflower extract, evening primrose extract, sea fennel extract, Rainbow algae extract, chamomile extract, oat extract, hyaluronic acid, and any combination thereof.

4. The peptide microparticle structure of claim 1, wherein the first lipid layer is made of at least one lipid.

5. The peptide microparticle structure of claim 1, wherein the second lipid layer is made of at least one lipid.

6. The peptide microparticle structure of claim 4, wherein the lipid consists of one of lecithin, phospholipid choline, cholesterol, and any combination thereof.

7. The peptide microparticle structure of claim 5, wherein the lipid consists of one of lecithin, phospholipid choline, cholesterol, and any combination thereof.

8. The peptide microparticle structure of claim 1, wherein the first lipid layer has a monolayer membrane structure or a bilayer membrane structure.

9. The peptide microparticle structure of claim 1, wherein the second lipid layer has a monolayer membrane structure or a bilayer membrane structure.

10. The peptide microparticle structure of claim 1, wherein the size of the peptide microparticle structure is 50-500 nm.
